(19) United States
(12) Patent Application Publication
(10) Pub. No.: US 2010/0312221 A1
(43) Pub. Date: Dec. 9, 2010

(54) WEARABLE DRUG DELIVERY DEVICE

(75) Inventors: Giovanni Nisato, Eindhoven (NL); Jan-Eric Jack Martijn Rubingh, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/600,010

(22) PCT Filed: May 20, 2008
(86) PCT No.: PCT/IB08/51974

§ 371 Date: Nov. 13, 2009

(30) Foreign Application Priority Data
May 22, 2007 (EP) 07108594.8

(51) Int. Cl. A61M 5/30

(52) U.S. Cl. 604/500; 604/70

(57) ABSTRACT

In order to provide a wearable drug delivery device for long term administration of drugs not employing a needle or canula, it is suggested according to the present invention that a wearable drug delivery device comprises a tubular reservoir (1) having an outlet end from which a drug may be expelled and a second end, a high-speed jet pump (2) for transdermal, needle-less micro-jet drug delivery, being connected to the outlet end of the tubular reservoir, a venting valve, being connected to the second end of the reservoir.

### FIELD OF THE INVENTION

The invention relates to the field of wearable drug delivery devices.

### BACKGROUND OF THE INVENTION

While oral delivery is the most common standard for drug delivery, many drugs cannot easily be formulated in a format suitable for oral administration. For example, treatment of diabetes, genetic disorders, and novel cancer treatments are based on (poly)peptides, which are destroyed in the gastro-intestinal tract. For these drugs, the preferred way of administration is usually an injection, and appropriate formulations need to be developed or matched to optimize the therapeutic effects, which can be highly dependent on the patient and can additionally be time-dependent. Furthermore, compliance is considered a major issue for the effective treatment of diseases. Therefore, there is a need for an alternative administration of drugs which provides an application of the right amount of drugs at the right time without requiring any action by the patient.

U.S. Pat. No. 4,734,092 discloses a device for infusing a drug into an ambulatory patient, the drug being contained in a transparent spiral conduit which is embedded in a disposable flexible casting conformingly adhered to the patient's body, includes a reusable micro-pump module, which is detachably mounted in a collar on the casting and forces oxygen into the conduit under pressure to expel the drug into a semi-pivoting canula inserted into the patient's body. A colored oil drop between the oxygen and the drug in the conduit provides a visual indication of drug quantity, while a filter of hydrophobic and hydrophilic membranes keeps the oxygen and oil substantially out of the canula. A test button sounds an alarm when the device is ready for use and a pressure sensitive switch automatically sounds an alarm and shuts off the pump if the drug becomes completely discharged from the conduit or if the drug delivery system becomes occluded and an interlock switch completes the circuit between the pump and a power source when the reusable module and disposable casting are joined.

The usage of a canula or needle requires the penetration of the patient's skin by the needle in order to administer the drug through the skin barrier. However, any entering of the canula to the patient's skin restricts the mobility and comfort of the patient.

### SUMMARY OF THE INVENTION

It would be advantageous to provide a wearable drug delivery device which would not require a canula for the application of the drug into the patient's body.

It would also be desirable to provide a wearable drug delivery device enhancing the mobility and comfort of the patient.

Furthermore it is desirable to provide a wearable drug delivery device according to an embodiment of the present invention does not require any surgical intervention for implantation of the device prior to the usage of the device.

It would also be desirable to provide a wearable drug delivery device operable when oriented in different directions, e.g. when the patient is standing up, lying down and having different orientations.

To better address one or more of these concerns, in a first aspect of the invention a wearable drug delivery device is provided comprising a tubular reservoir having an outlet end from which a drug may be expelled and a second end, a high-speed jet pump for transdermal, needle-less micro jet drug delivery, being connected to the outlet end of the tubular reservoir, a venting valve, being connected to the second end of the reservoir.

When compared on the other hand to needle-based drug delivery devices such as a syringe, the wearable drug delivery device according to an embodiment of the present invention does not require penetration of a needle or cannula into the patient.

Transdermal drug delivery, i.e. drug delivery directly through the skin, can be used for controlled and/or continuous delivery of drugs. Skin is an essential organ ensuring both protection from external pathogens and preventing water loss. In both cases, the barrier properties of skin, which are the result of millions of years of biological evolution, are essential to our survival. The top layer of the skin is the stratum corneum), the main layer ensuring barrier properties of the skin, which essentially consists of dead cells (corneocyte) surrounded by lipid bilayers. Due to their respective composition and structures, the stratum corneum is mostly hydrophobic and impermeable while the lower layers, epidermis and dermis, are mostly hydrophilic. As a consequence, molecules with low molecular weight of less than 5 kilo Dalton (kDa) and with a lipophilic character tend to permeate the skin rather than large, hydrophilic molecules.

According to an embodiment of the invention the high-speed jet pump for transdermal, needle-less micro jet drug delivery is a high-speed jet pump as disclosed in European patent application no. 06 119 215, the disclosure of which is incorporated herein in its full entirety by reference.

According to an embodiment of the invention, the high-speed jet pump comprises a casing with a fluid chamber, a membrane forming a wall of the fluid chamber, the fluid chamber further comprising at least one exit orifice and the membrane being piezo-electrically actuable for fluid ejection from the fluid chamber through the exit orifice, wherein a speed of the fluid ejection is adjustable by controlling the piezo-electric actuation of the membrane. Particularly in an embodiment of the present invention, the high-speed jet pump is an electrically driven needless injection device based on piezo-electric actuation.

In an alternative embodiment the high-speed jet pump may be based on an inductive coil actuating mechanism or any other high speed actuating mechanism. It is an advantage of a high-speed jet pump according to an embodiment of the present invention, that it allows the delivery of small amounts of the drug per injection.

It will be appreciated by a person skilled in the art, that the speed of the fluid ejection in an embodiment may advantageously be set to any desired value, for example depending on how deep into the patient's skin the fluid shall be delivered. The speed of the fluid ejection may as well be reduced below values at which the human skin is ruptured which advantageously allows ingestible or inplantable devices.

In a further embodiment, the speed of the fluid ejection is adjustable to a high-speed regime, and at least one dispensing regime, advantageously the high-speed jet pump according to an embodiment can be used both to pierce the epidermis, for example for transdermal drug delivery and to deliver controlled amounts of drug. The fluid ejection speed in the high speed regime is thus preferably at least sufficient for injecting the fluid through at least an outer layer of the skin of a patient. The top layer of the skin is the stratum corneum (sc), the main layer ensuring barrier properties of the skin. The fluid to be ejected is accelerated to an ejection speed high enough to disrupt the stratum corneum, to penetrate and diffuse in the epidermis and dermis, accessing peripheral blood vessels.

In an embodiment of the present invention, the fluid ejection speed in the high-speed regime is controllable, particularly between 60 m/s and 200 m/s. Therefore, the high-speed jet pump provides a broad range for utilization. The fluid ejection speed of 60 m/s is a typical speed for damage of soft tissue of biological nature such as bacterial films. A preferable fluid ejection speed by application in an embodiment according to the present invention in the high-speed regime for needle-less drug injection is about 20 m/s to 150 m/s.

In terms of the present invention, a wearable drug delivery device is a device which is arranged such that it can be carried by a patient in an operable condition on a long-term basis. Therefore, in a further embodiment of the wearable drug delivery device, it comprises mounting means for mounting the drug delivery device to a patient. Such mounting means could be self-adherent surfaces, bandages or strips to strap the device to the patient but are not restricted to such.

As the high-speed jet pump is used to eject the liquid drug through the patient's skin without puncturing the skin by a needle, it is essential that in the system consisting of the venting valve, the high-speed jet pump and the tubular reservoir being in fluid communication with each other, the jet pump is located as close as possible to the patient's skin, i.e. at the first of the tubular reservoir facing to the patient, from which the drug is expelled.

In comparison in the above reference the cannula or needle is connected to an outlet end of the spiral conduit, while the pump is connected to a second end of the conduit. When in operation the pump presses air into the first second end of the conduit, and therefore it expels the drug from the outlet end into the canula and into the patient's body.

A tubular reservoir in the terms of the present invention is a reservoir whose dimension in a first direction is at least twice as large as its dimension in the second direction.

The tubular reservoir according to an embodiment of the present invention at each filling level of the drug in the tubular reservoir has a minimal surface, i.e. the surface of the liquid level in the tube. Only the surface of the liquid in the tube forms the working surface for the external pressure.

In an embodiment of the invention the diameter and maximum radius of the tubular reservoir are adjusted to the properties drug solution to be injected so that the fluid is constrained in the tubular reservoir by capillary action. The parameters of interest are the surface tension γ of the fluid, the contact angle θ with the reservoir walls, the density ρ of the solution, the diameter of the reservoir, the maximum outer radius of the spiral manifold lₘₐₓ.

It is preferable that in an embodiment the internal diameter of the reservoir be less than dₘₐₓ, defined as:

$${d}_{max} = \frac{2 γcosθ}{πρ {gl}_{max}} .$$

This condition insures that the fluid does not leak out of the open nozzle or outlet orifice of the jet injector.

Furthermore, the tubular reservoir in an embodiment enables the usage of capillary forces keeping the fluid entirely between the filling level of an outlet orifice of the jet pump avoiding gas, e.g. air, to be pumped into the patient's body.

In an embodiment of the present invention, the medical grade tubing material should not interact chemically with the drug solution and should be sterilized prior to use. Tubing materials for the tubular reservoir include, but are not restricted to: polycarbonates, high-density polyethylene, nylon, retains, polypropylene, polyethylene, cyclic polyolefins, and the materials can be coated with inorganic compounds (e.g. silicon oxide) to reduce the contact angle θ for aqueous solutions. The tubing material in an embodiment can be transparent to allow for optical inspection, a fluid level monitoring and to detect the presence of air bubbles in the tubular reservoir.

The tubing inner diameter in an embodiment ranges from 0.4 mm to 2 mm. In an embodiment, the volumes available for the fluid storage are in the range from 1 to 5 ml.

The overall volume of the reservoir in an embodiment is smaller than 10 ml. Preferably, the construction of the tubular reservoir is flexible such that it can occupy the volume of a casing in an optimum manner.

In an embodiment of a present invention, the venting valve is located adjacent to the jet pump. “Adjacent” herein means that the venting valve is located close to the nozzle or outlet orifice of the high-speed jet pump, in order to reduce the possible hydrostatic pressure differences between the venting valve and the outlet orifice of the jet pump as much as possible. This way, the differences in hydrostatic pressure between the venting valve and the micro jet pump can be minimized.

In a further embodiment, the distance between the jet pump and the venting valve is smaller than 2 cm and preferably smaller or equal to 1 cm.

Desirably, there is an embodiment of the invention in which the tubular reservoir is spirally arranged. A spiral arrangement as understood in terms of the present invention requires that at least part of the tubular reservoir forms a spiral such that when being pressed through the tubular reservoir, the liquid drug moves inward or outward on a spiral track. This construction can minimize the differences in hydrostatic pressure between the reservoir and the jet pump and enables the application of the drug in every different physical orientation of the patient and thus of the wearable drug delivery device according to an embodiment of the present invention. In an alternative embodiment of the present invention, the spiral formed by the tubular reservoir is arranged essentially in a plane and the venting valve and the jet pump are arranged on an axis perpendicular to the plane.

Furthermore, an embodiment of the invention may be advantageous in which the jet pump and the venting valve are arranged in the center of the spirally arranged tubular reservoir.

In an embodiment, the venting valve comprises a semi-permeable membrane fastened at the second end of the tubular reservoir, wherein the membrane works as a sealing for any liquids and is permeable for gas, i.e. air.

In a further alternative embodiment of the present invention, the wearable drug delivery device comprises a filling system enabling a refilling of the reservoir while being attached to the patient's body.

In an embodiment, the reservoir may be refilled through the filling system using a standard syringe with a hypodermic needle. Therefore in an embodiment, the filling system comprises a septum forming one of its outer walls, in which the hypodermic needle of the syringe may be inserted.

In order to avoid the injection of air through the filling system into the tubular reservoir, the filling system in an embodiment may comprise an electrical or optical system enabling the detection of gas bubbles in the liquid drug being injected into the filling system.

Alternatively, in an embodiment of the wearable drug delivery device refilling may be achieved through the outlet orifice or nozzle of the jet pump.

According to a further embodiment of the invention, the filling system is in fluid communication with the tubular reservoir such that it divides the tubular reservoir in two sections. This design may enable a bubble-free ejection of the liquid drug from the high-speed jet pump during operation of the device.

These and other aspects of the invention will be apparent from and elucidated with reference to the embodiments described hereinafter.

### BRIEF DESCRIPTION OF EMBODIMENTS

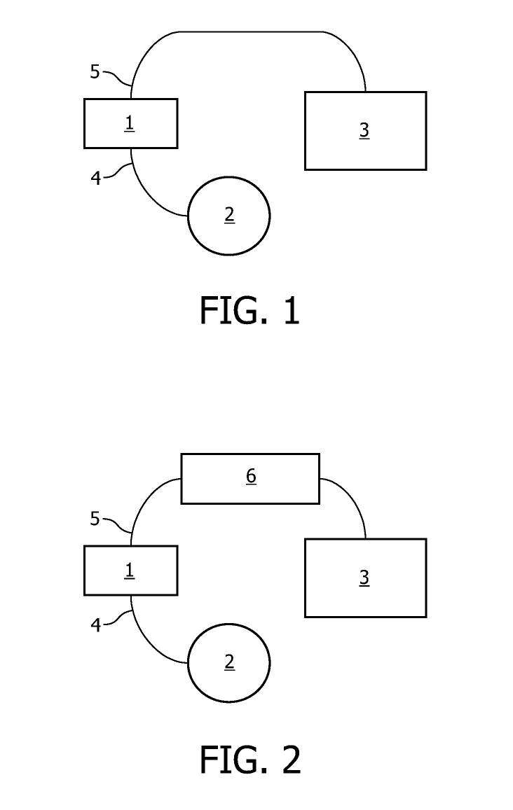

FIG. 1 diagrammatically shows a first embodiment of a wearable drug delivery device according to the present invention.

FIG. 2 diagrammatically shows a further embodiment of a wearable drug delivery device according to the present invention.

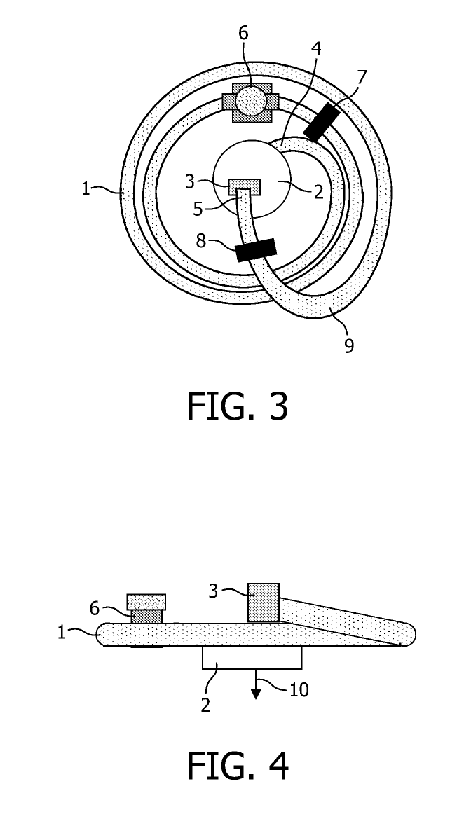

FIG. 3 shows a top view of a wearable drug delivery device according to the embodiment shown in FIG. 2.

FIG. 4 shows a side view of the embodiment of FIG. 3.

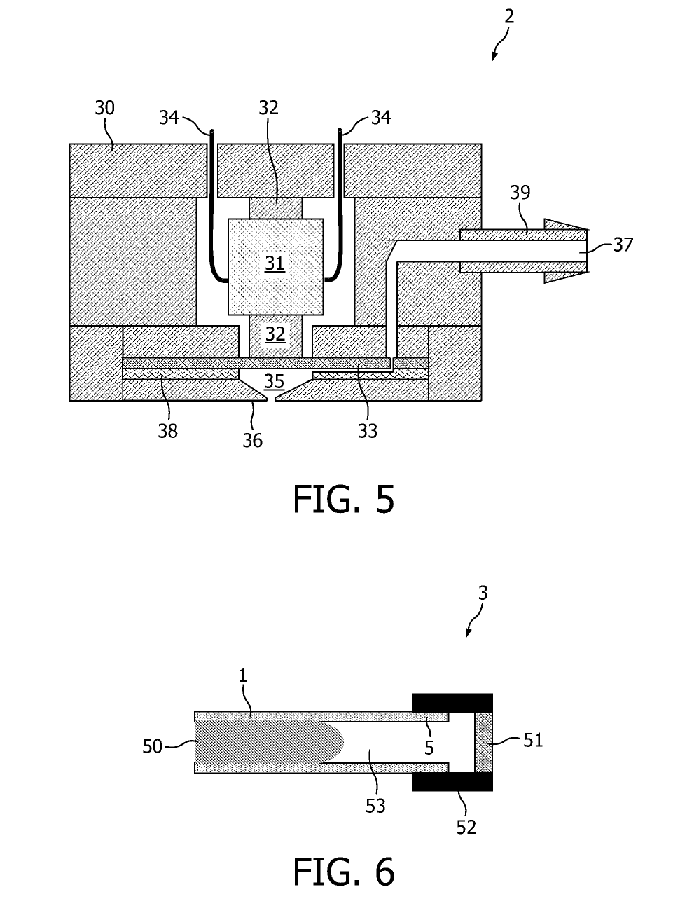

FIG. 5 shows a schematic cross-sectional view of a high-speed piezo jet pump being part of the device shown in FIGS. 3 and 4.

FIG. 6 shows a cross-sectional view of the venting valve being part of the device shown in FIGS. 3 and 4.

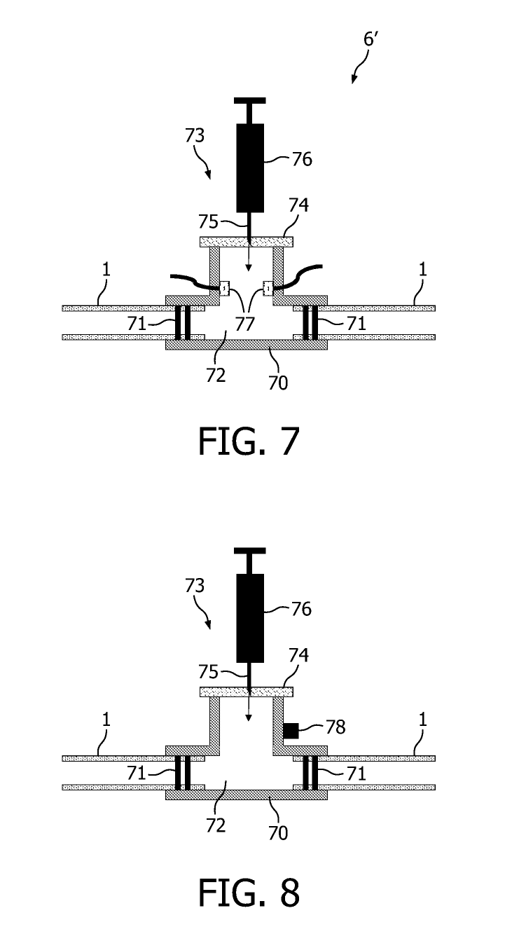

FIG. 7 shows a first embodiment of a filling system.

FIG. 8 shows a second embodiment of a filling system.

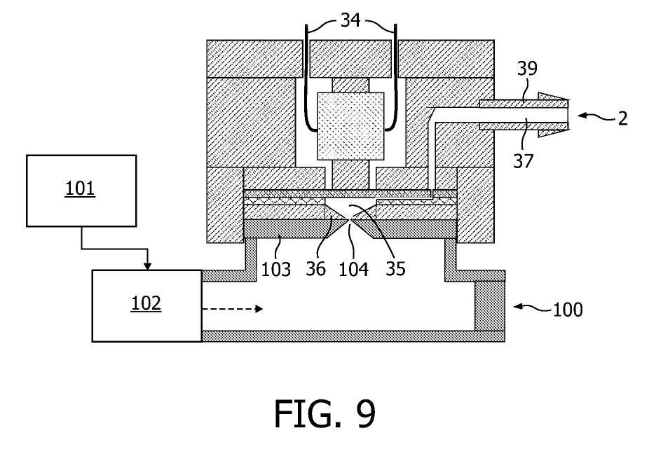

FIG. 9 shows an alternative filling system.

### DETAILED DESCRIPTION OF EMBODIMENTS

FIG. 1 schematically shows the components of a wearable drug delivery system according to a first embodiment comprising a tubular reservoir 1, a high-speed jet pump 2 as well as a venting valve 3. The three components 1, 2, 3 are in fluid communication with each other, i.e. a liquid drug may flow or may be pumped from the venting valve 3 via the tubular reservoir 1 to the jet pump 2. The tubular reservoir 1 comprises an outlet end 4 and a second end 5. The outlet end 4 of the tubular reservoir 1 is considered the end from which a dug is ejected through the pump 2 into a patient's body. The jet pump 2 is connected to the outlet end 4 of a tubular reservoir 1. In contrast, the venting valve 3 is connected to the second end 5 of the tubular reservoir 1.

In FIG. 2, a further alternative embodiment of a wearable drug delivery device according to the present invention is schematically drawn. If compared to FIG. 1, the device as laid out in FIG. 2 further comprises a filling system 6 enabling a refilling of the tubular reservoir 1.

FIG. 3 shows a top view on a system as schematically laid out in FIG. 2 comprising a tubular reservoir 1, a high-speed jet pump 2, a venting valve 3 and a filling system 6. Further to the components shown schematically in FIG. 2, the wearable drug delivery device shown in FIG. 3 has two filling sensors 7, 8.

From the top view in FIG. 3, it will be appreciated by a person skilled in the art how the tubular reservoir in an embodiment is arranged in order to provide full functionality. The tubular reservoir 1 extends from an outlet end 4 being connected to the jet pump 2 to a second end 5 being connected to the venting valve. Starting from the outlet end 4, the tubular reservoir 1 is arranged in a spiral winding radially outward on a spirally shaped track.

In the embodiment shown, the tubular reservoir is made of transparent Teflon having an inner diameter of 0.75 mm. The overall volume of the tubular reservoir 1 is 5 ml. Before reaching the second end 5 of the tubular reservoir 1 carrying the venting valve 3, the tubular reservoir reaches a point 9, from which onward the tubular reservoir no longer extends on a spiral track, but bends inwardly towards the center of the spiral. In the embodiment shown in FIGS. 3 and 4, the jet pump 2 as well as the venting valve 3 are positioned approximately at the center of the spirally shaped tubular reservoir 1.

As the outlet end 4 and the second end 5 of the tubular reservoir 1 and the jet pump 2 and the venting valve 3, respectively, are arranged adjacent to each other in close proximity, hardly any differences in hydrostatic pressure between the jet pump 2 and the venting valve 3 of the tubular reservoir occur.

This arrangement of the venting valve 3 and the jet pump 2 being close proximity to each other can be further be understood from FIG. 4, which shows a side view of the wearable drug delivery device depicted in FIG. 3. Denoted by reference number 10 is an arrow indicating the direction of a fluid beam being ejected from the nozzle of the jet pump 2. Although horizontally slightly separated, the jet pump 2 and the venting valve 3 lie together on a line defined by the arrow 10 representing the direction of a fluid beam being ejected from the jet pump 2.

FIG. 5 shows an elaborate cross-sectional view of the jet pump 2 as used in the embodiment of FIGS. 3 and 4. In FIG. 5, the jet pump 2 is schematically depicted in cross-section comprising a casing 30, a piezo-electric transducer 31, mechanically coupled via support structure 32 to the casing 30 at a first site and to a membrane 33 at the other site. The piezo-electric transducer 31, for example a small bulk piezo-electric transducer of multi-layer ceramic is driven via powerlines 34, which connect the piezo-electric transducer 31 to a driving unit (not shown). A micro-controller controls the pump, in particular the supply of the piezo-electric transducer 31. The membrane 33 forms a wall of a fluid chamber 35 which comprises an outlet orifice or a nozzle 36 and which is connected to a fluid supply line 37. The fluid supply line 37 leads through the membrane 33 remote from the fluid chamber and runs at least partly between the membrane 33 and in interlayer 38. Fluid is supplied to the device via an intake connection 39 which is located at one side of the device. The intake connection 39 is connected to the outlet end 4 of the tubular reservoir 1 as shown in FIGS. 3 and 4.

During driving of the piezo-electric transducer 31, the piezo-electric transducer 31 expands and pushes on the flexible membrane 33. This compresses the fluid in the fluid chamber 35, resulting in a pressure built up and as a consequence, a fluid flow out of the exist orifice 36. The exit orifice 36 is formed as a nozzle with a diameter typically ranging from 10 μm to 200 μm and a length between 50 μm and 200 μm. As soon as the driving of the piezo-electric transducer 31 stops, both the piezo-electric transducer 31 and the membrane 33 return to their rest state and fluid will enter the fluid chamber 35 through the fluid supply line 37 by capillary force.

In order to generate a high-speed fluid ejection, the high-speed jet pump as used in the embodiments shown is mechanically stiff. If there was too much mechanical deformation of the device during driving of the piezo-electric transducer 31, the pressure in the fluid chamber would be too low to generate a high-speed fluid ejection. Further, the relation between the length and diameter of the fluid supply line 37 and the length and diameter of the nozzle 36 determine the functioning of the employed jet pump.

FIG. 6 shows a schematic cross-sectional view of the venting valve 3 as employed in the wearable drug delivery device shown in FIGS. 3 and 4. FIG. 6 shows the second end 5 of the tubular reservoir 1 containing the liquid drug 50 and air 53, the venting valve 3 comprising a mount 52 and a semi-permeable membrane 51 sealing the second end 5 of the tubular reservoir 1. The semi-permeable membrane 51 mounted by the mount 52 is permeable for gases like air and provides a solid barrier for a fluid like the drug 50 in the reservoir 1. Therefore, air 53 enclosed in the tubular reservoir 1 can degas through the semi-permeable membrane 51 when the reservoir 1 is filled through the filling system 6 with a liquid drug 50. On the other hand, the semi-permeable membrane provides a venting, i.e. a flow of air into the reservoir 1 when the fluid 50 is ejected by the jet pump 2 from the tubular reservoir 1 avoiding the built up of a vacuum in the tubular reservoir counter-acting on the pumping forces of the jet pump 2.

FIGS. 7, 8 and 9 show alternative embodiments of a filling system enabling a refilling of a liquid drug into the tubular reservoir 1. While FIGS. 7 and 8 do show two different embodiments of the filling system 6 as depicted in FIGS. 2 to 4, FIG. 9 shows an external filling system allowing a refill of the wearable drug delivery system according to FIG. 1.

FIG. 7 shows a first embodiment of a filling system 6′ as shown e.g. in FIG. 3, the filling systems 6, 6′, 6″ are preferably arranged at a location in between the outlet end 4 and the second end 5 of the tubular reservoir 1. Therefore, the filling systems 6, 6′, 6″ as depicted in FIGS. 7 and 8, do have a casing 70 mounted with O-rings 71 to two sections of the tubular reservoir 1, whereas one section of the tubular reservoir 1 leads to the outlet end being connected to the jet pump 2, the second section leads to the second end 5 being connected to the venting valve 3. The casing 70 provides a chamber 72 for a fluid flow from the first section to the second section of the tubular reservoir and an inlet orifice 73 being sealed by a septum 74. The tubular reservoir 1 may be refilled using a standard syringe with a hypodermic needle. The hypodermic needle is inserted into the filling system 6′, 6″ by punching the hypodermic needle 75 of the syringe 76 through the septum 74.

While the embodiment of the filling system 6′ shown in FIG. 7 comprises two ohmic fluid sensors 77, the second embodiment of the filling system 6″ shown in FIG. 8 comprises an optical fluid sensor 78. In each case, the fluid sensors 77, 78 allow for the detection of bubbles, which may be injected from the syringe via the hypodermic needle 75 into the tubular reservoir 1. In case any of the fluid sensors 77, 78 detect a gas bubble flowing through the inlet orifice 73 of the filling system 6′, 6″, a warning signal is provided to a central controller (not shown).

FIG. 9 shows an alternative external filling system 100 being coupled to a jet pump 2 as described in detail above. The external filling system 100 provides an alternative way of refilling the tubular reservoir 1 replacing the filling systems 6′, 6″ as depicted in FIGS. 7 and 8. The filling system 100 is therefore well-suited to an embodiment as depicted in FIG. 1. The filling system 100 comprises a reservoir 1 containing the drug, a pump 102 and a coupling portion 103 having an orifice 104 matching the nozzle 36 of the pump 2. In operation, the drug is then pumped by the pump 102 from the reservoir 101 into the coupling portion 103 and through the outlet orifice through the nozzle 36 of the fluid chamber 35, the fluid intake 37 and the intake connection 39 of the pump 2 into the tubular reservoir 1.

While the invention has been illustrated and described in detail in the drawings and foregoing description, such illustration and description are to be considered illustrative or exemplary and not restrictive, the invention is not limited to the disclosed embodiments.

Other variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. The mere fact that certain measures are recited in mutually different dependent claims does not indicate that the combination of these measures cannot be used to an advantage. Any reference signs in the claims should not be construed as limiting the scope.

### LIST OF REFERENCE NUMERALS

1 Tubular reservoir
2 Jet pump
3 Venting valve
4 Outlet end of the tubular reservoir
5 Second end of the tubular reservoir
6, 6′, 6″ Filling system
7, 8 Filling sensors
9 Bending of the reservoir
10 Arrow indicating the direction of a fluid beam
30 Casing
31 Piezo-electric transducer
32 Support structure
33 Membrane
34 Powerlines
35 Fluid chamber
36 Nozzle
37 Fluid supply line
38 Interlayer
39 Intake connection
50 Liquid drug
51 Semi-permeable membrane
52 Mount
53 Air
70 Casing
71 O-rings
72 Chamber for first fluid flow
73 Inlet orifice
74 Septum
75 Hypodermic needle
76 Syringe
77 Ohmic fluid sensors
78 Optical fluid sensor
100 Alternative external filling system
101 Reservoir
102 Pump
103 Coupling portion
104 Orifice

1. A wearable drug delivery device comprising
a tubular reservoir (1) having an outlet end (4) from which a drug may be expelled and a second end (5),
a high-speed jet pump (2) for transdermal, needle-less micro jet drug delivery, being connected to the outlet end (4) of the tubular reservoir (1),
a venting valve (3), being connected to the second end (5) of the reservoir (1).

2. A wearable drug delivery device according to claim 1, wherein the venting valve (3) is located adjacent to the jet pump (2).

3. A wearable drug delivery device according to claim 1, characterized in that the tubular reservoir (1) is spirally arranged.

4. A wearable drug delivery device according to claim 3, characterized in that the spiral formed by the tubular reservoir (1) is arranged in a plane and wherein the venting valve (3) and the jet pump (2) are arranged on a common axis perpendicular to the plane.

5. A wearable drug delivery device according to claim 3, characterized in that the jet pump (2) and the venting valve (3) are arranged in the center of the spirally arranged tubular reservoir (1).

6. A wearable drug delivery device according to claim 1, characterized in that it comprises a filling system (6, 6′, 6″).

7. A wearable drug delivery device according to claim 6, characterized in that the filling system (6, 6′, 6″) is in fluid communication with the tubular reservoir (1) such that it divides the tubular reservoir (1) in two sections.

8. A wearable drug delivery device according to claim 1, characterized in that it comprises mounting means for mounting the drug delivery device to a patient.

9. Method for the administration of a drug using a wearable drug delivery device comprising
a tubular reservoir (1) having a first outlet end (4) from which a drug may be expelled and a second end (5),
a high-speed jet pump (2) for transdermal, needle-less micro jet drug delivery, being connected to the outlet end (4) of the tubular reservoir (1),
a venting valve (3), being connected to the second end (5) of the reservoir (1).
